(19) United States
(12) Patent Application Publication
(10) Pub. No.: US 2024/0260581 A1
(43) Pub. Date: Aug. 8, 2024

(54) ANTIMICROBIAL COMPOSITION, METHOD FOR PRODUCING AN ANTIMICROBIAL COMPOSITION AND USE OF AN ANTIMICROBIAL COMPOSITION

(71) Applicant: FNT-GMBH, Rudersberg (DE)

(72) Inventor: Volker MÜHLBERGER, Rutesheim (DE)

(73) Assignee: FNT-GMBH, Rudersberg (DE)

(21) Appl. No.: 18/563,018

(22) PCT Filed: Feb. 16, 2022
(86) PCT No.: PCT/DE2022/200023

§ 371 Date: Nov. 21, 2023

(30) Foreign Application Priority Data
May 28, 2021 (DE) 10 2021 205 475.7

(51) Int. Cl. A01N 59/16; A01N 25/04; A01N 25/22; A01P 1/00; A61K 33/38; A61P 31/04; A61P 31/10

(52) U.S. Cl. CPC A01N 59/16; A01N 25/04; A01N 25/22; A01P 1/00; A61K 33/38; A61P 31/04; A61P 31/10

(57) ABSTRACT

An antimicrobial composition is described, as well as a method for preparing an antimicrobial composition and the use of an antimicrobial composition.

The invention relates to an antimicrobial composition, a method for producing the antimicrobial composition, and the use of the antimicrobial composition.

The antimicrobial effects of silver have long been known. Already in the Middle Ages it was found that milk, for example, lasted longer if a silver coin was added to it. In medicine, ulcers have long been treated with silver nitrate. The antimicrobial effect results from the silver ions that are released from elemental silver or that are formed when silver nitrate is dissolved in water. The silver ions are absorbed by microorganisms or attach themselves to them. This disrupts the metabolism and reproduction of microorganisms and causes them to die.

In the context of nanotechnology, silver has recently been used in nanoparticulate form. These are silver particles that typically do not exceed 100 nm in diameter. Methods for the production of silver nanoparticles are known from the prior state of the art. However, the particle diameter and/or the concentration of the silver particles is limited in these methods, as the particles become unstable during their build-up due to mutual interactions or interfering substances.

The antimicrobial activity of silver nanoparticles results from the increased surface area and the associated increased release of silver ions. Silver nanoparticles are used, for example, for the coating of catheters or implants. Nanoparticulate silver is also used in wound dressings to cover burns or cuts, among other things. In ointments it is used for the treatment of allergic contact dermatitis or eczema. Contaminated drinking water can be purified with filters containing silver nanoparticles. Nanoparticulate silver is used in clothing to kill odour-causing bacteria. In addition to the antibacterial effect, silver nanoparticles also exhibit antiviral and antifungal effects.

The disadvantage is that microorganisms can occur that are resistant to silver ions. There is also the possibility that if the amount of silver ions released is too high, the material may be toxic to humans. It is also not ruled out that nanoparticles can pass into the gas phase via solvents or water vapour. There is therefore a risk that the nanoparticles will penetrate the body through the skin due to their small size. The nanoparticles could also be inhaled and enter the body via the lungs. Therefore, in addition to the current wide range of applications of nanoparticulate silver, there is also a strong focus on the possible health risks.

The present invention is therefore based on the object of providing an antimicrobial composition and a method for producing an antimicrobial composition which overcome the above-mentioned problems.

With this invention as described here, the aforesaid task is solved by the features of claim 1. Next, the invention provides an antimicrobial composition comprising colloidal silver and at least one stabiliser that stabilises the colloidal silver, wherein the colloidal silver has a particle diameter in the range of 700 nm to 9000 nm.

An antimicrobial composition is to be understood as meaning a composition that inhibits the growth of microorganisms. This can be done by killing the microorganisms directly or by preventing them from multiplying further.

The term “microorganisms” refers to bacteria, fungi and viruses.

The term “colloid” refers to a dispersion of particles of one substance in another substance. What is characteristic of colloids is that the dispersed particles are so small that in some respects they behave like dissolved molecules; for example, they do not sediment. On the other hand, however, they have properties of discrete particles with boundary surfaces. The colloid particles themselves are called the disperse phase, the surrounding phase that absorbs the particles is called the dispersant.

Accordingly, the term “colloidal silver” used here refers to particles of elemental silver or their liquid dispersions.

By definition, the colloidal silver particles in the composition according to the invention do not belong to the nanoparticles, which usually do not exceed a diameter of 100 nm. The colloidal silver particles in the composition described here are not water vapour volatile and therefore cannot be inhaled. In addition, their size prevents them from being absorbed into the body through the (mucous) skin. Furthermore, the surface area is reduced compared to nanoparticulate silver, which means that only a moderate amount of silver ions are released.

Consequently, the risk of silver ions having a toxic effect on the body is reduced.

In a further embodiment, the colloidal silver has a particle diameter of 750 nm to 7000 nm. Preferably the particle diameter is in the range from 800 nm to 5000 nm. The particle diameter is preferably in the range from 850 nm to 3000 nm, preferably in the range from 900 nm to 2000 nm, preferably in the range from 950 nm to 1500 nm. The particle diameter of colloidal silver can be 700 nm, 800 nm, 900 nm, 1000 nm, 1100 nm, 1200 nm, 1300 nm, 1400 nm, 1500 nm, 2000 nm, 2500 nm, 3000 nm, 3500 nm, 4000 nm, 4500 nm, 5000 nm, 5500 nm, 6000 nm, 6500 nm, 7000 nm, 7500 nm, 8000 nm, 8500 nm or 9000 nm.

In principle, colloids are thermodynamically unstable compared to the corresponding volume phases because of their large surface area. This means that the particles tend to reduce their specific surface area through aggregation. To prevent aggregation of the colloidal particles from forming during their build-up, so-called stabilisers are used. A distinction is made between stabilisation of the particle surface through electrostatic repulsion and through steric hindrance. In electrostatic repulsion, the adsorption of ions on the particle surface and the formation of an electrostatic double layer stabilises the particles, which then results in electrostatic repulsion between the individual particles and prevents aggregation. Stabilisation of the particles by steric hindrance can be achieved by the stabiliser separating the individual particles from each other so that the necessary approach between the particles for aggregation is prevented.

Possible stabilisers include, for example, agar and/or dispersions of silicon dioxide particles. Agar is a galactose polymer that has a solid gel-like consistency when cooled. Dispersions of silicon dioxide particles are colloidal silicon dioxide particles. The silicon dioxide particles can have different surface charges depending on the pH. In an alkaline pH range, the silicon dioxide particles are preferably present with a negative surface charge. These are therefore anionic silicon dioxide particles. In an acidic pH range, the silicon dioxide particles are preferably present with a positive surface charge. These are cationic silicon dioxide particles.

In a preferred embodiment, the stabiliser is agar. Agar has the advantage that it does not become unstable in either acidic or alkaline pH. In addition, the particle sizes of the agar can be adjusted by simply crushing it. Agar is also non-toxic, tasteless and biodegradable, which underlines the environmental compatibility of the composition. In addition, agar is approved for use in food.

In a further preferred embodiment, the stabiliser is a dispersion of silicon dioxide particles. Silicon dioxide has the advantage that it can be dispersed in a liquid. The silicon dioxide particles can thus be evenly distributed in the liquid, which leads to improved stabilisation of the colloidal silver. Depending on the pH, the silicon dioxide particles gel. This also makes it possible to regulate the particle sizes of the silicon dioxide in the dispersion. Moreover, silicon dioxide is non-toxic and insoluble in water. It can therefore not be inhaled through the air and poses no health risk.

Another advantage of silicon dioxide is that it has photocatalytic properties. This refers to the generation of reactive oxygen species through the absorption of electromagnetic radiation in the UV range. For example, in combination with water, hydrogen peroxide is formed via photocatalysis. The reactive oxygen species damage the microorganisms so that they die.

In a further embodiment, the stabiliser is a combination of agar and a dispersion of silicon dioxide particles. By using agar and a dispersion of silicon dioxide particles as stabilisers, the respective advantages of the individual stabilisers are combined, which has a synergistic effect on the stability of the colloidal silver particles.

In one embodiment, the dispersed silicon dioxide particles are present in the composition at a concentration ranging from 1 g/l to 5 g/l. Preferably, the silicon dioxide particles are present in the composition in a concentration in the range from 1.25 g/l to 4 g/l, preferably in a concentration in the range from 1.5 g/l to 3 g/l. In the antimicrobial composition, the dispersed silicon dioxide particles can therefore be in a concentration of 1 g/l, 1.25 g/l, 1.5 g/l, 1.75 g/l, 2 g/l, 2.25 g/l, 2.5 g/l, 2.75 g/l, 3 g/l, 3.25 g/l, 3.5 g/l, 3.75 g/l, 4 g/l, 4.25 g/l, 4.5 g/l, 4.75 g/l or 5 g/l.

It was recognised that dispersions with silicon dioxide particles that have a diameter of 700 nm to 900 nm stabilise the colloidal silver particularly well. Accordingly, in a further embodiment, the diameter of the dispersed silicon dioxide particles can be in the range from 700 nm to 900 nm. Preferably, the diameter of the dispersed silicon dioxide particles is in the range of 750 nm to 850 nm. In one embodiment, the dispersed silicon dioxide particles therefore have a diameter of 700 nm, 750 nm, 800 nm, 850 nm, or 900 nm.

Good results with regard to the stability of the colloidal silver can also be achieved if agar is used as a stabiliser and the agar is in particles with a diameter in the range of 9000 nm to 10000 nm. Preferably, the diameter of the agar particles is in the range from 9000 nm to 9500 nm. In one embodiment, the agar is therefore present as particles with a diameter in the range of 9000 nm to 10000 nm, preferably in a range of 9000 nm to 9500 nm. The agar particles can in particular have a diameter of 9000 nm, 9100 nm, 9200 nm, 9300 nm, 9400 nm, 9500 nm, 9600 nm, 9700 nm, 9800 nm, 9900 nm or 10000 nm. Particles of this size are pourable and can be easily produced using standard mixing devices.

In order to increase the duration of the antimicrobial effect of the composition on surfaces, the composition can be applied as a film to the relevant surface, with the film acting as a coating on the surface after drying. The coating can be permanent or temporary. Mechanical stress can cause the coating to be removed from the surface.

In order for the film to be present as a coating on the surface, a film-forming polymer dispersion can be added to the antimicrobial composition. The polymer dispersion must be stable to the components of the composition, have good drainage properties and must contain the dispersed components evenly distributed when drying. Polyethylene waxes, polyacrylates or polyurethanes can be used as polymer dispersions. Polyethylene waxes can, for example, be selected from polymers that are sold under the name Lugalvan® DC or Südranol® 220 SEC. The polymer that can be used as polyacrylate is sold under the name Alberdingk® AC2403. In addition, copolymers with polyurethane and polycarbonate units can be used in the composition. For example, a possible polyurethane-polycarbonate copolymer is the copolymer sold under the name Alberdingk® PU6800.

To ensure that the components of the composition can be mixed evenly with the polymer dispersion so that it can enclose the components during drying, a minimum amount of approximately 5% by weight of the polymer dispersion in the composition is necessary. The polymer dispersion can constitute up to 20% by weight of the composition. Accordingly, the composition may contain 5% by weight to 20% by weight of film-forming polymer dispersion. Preferably, the composition may comprise 10% to 15% by weight of the polymer dispersion. Accordingly, in a further embodiment, the polymer dispersion may be present at 5% by weight, 10% by weight, 15% by weight or 20% by weight in the antimicrobial composition.

In a further embodiment, the polymer dispersion can be used in the composition at a concentration in the range of 10 g/l to 80 g/l. Preferably, the polymer dispersion may be present in the composition in a range of 20 g/l to 70 g/l, preferably in a range of 30 g/l to 60 g/l.

In particular, the polymer dispersion can have a concentration of 10 g/l, 15 g/l, 20 g/l, 25 g/l, 30 g/l, 35 g/l, 40 g/l, 45 g/l, 50 g/1, 55 g/l, 60 g/l, 65 g/l, 70 g/l, 75 g/l or 80 g/l in the composition.

The weight ratio of the polymer dispersion to the composition can in particular be in the range from 1:5 to 5:1. Preferably the weight ratio of the polymer dispersion to the antimicrobial composition is in the range of 1:2 to 2:1. Preferably the weight ratio of polymer dispersion to the antimicrobial composition is 1:1.

The composition may further comprise at least one surfactant. Surfactants are responsible for optimum wetting of surfaces, especially hydrophobic surfaces such as plastics or glass. The surfactant lowers the surface tension of the composition, which increases wettability and helps distribute the composition evenly. The uniform distribution of the composition is necessary, for example, if the composition is to be applied to a surface as a film for, for example, a coating.

Non-ionic surfactants are particularly suitable for the composition according to the invention. Ethoxylated fatty alcohols are preferred as non-ionic surfactants. For example, decan-1-ols with a degree of ethoxylation of 1 to 10 can be used as ethoxylated fatty alcohols in the first liquid. Preferably, the surfactant can be decan-1-ol with a degree of ethoxylation of 5, which is sold under the brand name Zusolat 1005/85.

Therefore, in a further embodiment, the surfactant is a non-ionic surfactant. It is preferable for the surfactant to be an ethoxylated fatty alcohol.

In a further embodiment, the surfactant can be used in the composition at a concentration in the range of 1 ml/l to 5 ml/l. At a concentration of less than 1 ml/l, there is a risk that sufficient wetting of the surface cannot be guaranteed and the composition is only present locally and not as a coherent film on the surface. At a concentration above 5 ml/l, the risk of air inclusions due to increased foam formation increases. This can lead to uneven distribution of the individual components of the composition. There is also the possibility that a composition applied as a film may burst. Preferably, the surfactant can be used in the antimicrobial composition with a concentration in the range from 1.25 ml/l to 3 ml/l, preferably with a concentration in the range from 1.5 ml/l to 2 ml/l In particular, the concentration of the surfactant in the composition can be 1 ml/l, 1.25 ml/l, 1.5 ml/l, 1.75 ml/l, 2 ml/l, 2.5 ml/l, 3 ml/l, 3.5 ml/l, 4 ml/l, 4.5 ml/l or 5 ml/l.

To enhance the antimicrobial effect of the composition, titanium dioxide can be added to the composition. Titanium dioxide also has photocatalytic properties and can therefore contribute to the formation of reactive oxygen species.

The titanium dioxide can be in the form of a dispersion of titanium dioxide particles. The diameter of the titanium dioxide particles can be in a range from 1 μm to 10 μm, preferably in a range from 3 μm to 7 μm, preferably in a range from 4 μm to 6 μm. In particular, the diameter of the dispersed titanium dioxide particles can be 1 μm, 2 μm, 3 μm, 4 μm, 5 μm, 6 μm, 7 μm, 8 μm, 9 μm or 10 μm.

The titanium dioxide can be present in the composition in a concentration of 1 g/l to 5 g/l. At a concentration of less than 1 g/l, the photocatalytic effect of titanium dioxide is too low to have an additional effect on the microorganisms. At concentrations greater than 5 g/l, there is a risk that the dispersed titanium dioxide will settle and will therefore no longer be evenly distributed throughout the composition. In addition, the transparency of the composition is impaired, which is disadvantageous for coatings, especially on glass. In a further embodiment, the concentration of the dispersed titanium dioxide is in the range from 1 g/l to 5 g/l, preferably in the range from 2 g/l to 4 g/l. In particular, the titanium dioxide can be present in the composition in a concentration of 1 g/l, 2 g/l, 3 g/l, 4 g/l or 5 g/l.

Advantageously, in one embodiment, the colloidal silver is present in the antimicrobial composition in a concentration of over 100 ppm. Preferably the colloidal silver is present in a concentration of over 200 ppm. Further preferably, the colloidal silver is present in a concentration of over 300 ppm, preferably over 500 ppm. In one embodiment, the colloidal silver is present in the composition at a concentration of over 800 ppm, preferably over 1000 ppm.

Depending on the use of the antimicrobial composition, different concentrations of colloidal silver are necessary in order to develop a sufficient antimicrobial effect. In principle, a concentration of 100 ppm has proven to be sufficiently effective. At high concentrations, however, there is a risk that too many silver ions are released per unit of time. Economic reasons also play a role in the level of concentration. In one embodiment, the colloidal silver in the composition is present in a concentration in the range of 100 ppm to 1500 ppm. Preferably, the colloidal silver in the composition is present in a concentration in the range from 200 ppm to 1000 ppm. Further preferably, the concentration of colloidal silver is in the range of 300 ppm to 800 ppm, preferably in the range of 400 ppm to 600 ppm. There are conceivable uses that require highly concentrated colloidal silver. In such cases, the concentration of colloidal silver in the composition may be in the range of 1000 ppm to 1500 ppm, preferably in the range of 1200 ppm to 1300 ppm.

In a further embodiment, the colloidal silver may be present in a concentration of 100 ppm, 200 ppm, 300 ppm, 400 ppm, 500 ppm, 600 ppm, 700 ppm, 800 ppm, 900 ppm, 1000 ppm, 1100 ppm, 1200 ppm, 1300 ppm, 1400 ppm or 1500 ppm.

A further advantage of the composition according to the invention is that only small amounts of stabiliser need to be present in the composition to stabilise the colloidal silver. In one embodiment, the ratio of colloidal silver to silicon dioxide may range from 100 ppm colloidal silver per 300 mg of silicon dioxide to 100 ppm colloidal silver per 100 mg of silicon dioxide. Preferably, the ratio of colloidal silver to silicon dioxide is in the range of 100 ppm colloidal silver per 250 mg of silicon dioxide to 100 ppm colloidal silver per 125 mg of silicon dioxide.

A further advantage of the composition described here is that it does not contain any solvents. In addition, the components are not water vapour volatile. This means that the components cannot get into the ambient air and be inhaled with it and thus penetrate the body via the lungs. This avoids possible health risks to the body.

In a further embodiment, the antimicrobial composition comprises water, agar, a dispersion of silicon dioxide particles and colloidal silver.

In a further embodiment, the antimicrobial composition comprises water, a dispersion of silicon dioxide particles and colloidal silver.

In a further embodiment, the antimicrobial composition comprises water, a dispersion of silicon dioxide particles, colloidal silver, a film-forming polymer dispersion and a surfactant.

In a further embodiment, the antimicrobial composition may have a pH in the range of 6.5 to 7, preferably in the range of 6.7 to 6.8. In particular, the antimicrobial composition can have a pH of 6.5; 6.6; 6.7; 6.8; 6.9; or 7.

In a further embodiment, the antimicrobial composition may remain stable for at least 6 months, preferably for at least 12 months, preferably for at least 24 months, and maintain antimicrobial efficacy.

The described antimicrobial composition is suitable for a wide range of applications. In particular, the composition can be used to treat or prevent infestation with bacteria, fungi and/or viruses.

According to a further embodiment, the antimicrobial composition can be used in human and/or veterinary medicine. Preferably, the antimicrobial composition can be used for the treatment or prevention of superficial bacterial, fungal and/or viral diseases. For example, the composition can be applied to affected areas of the skin to treat them. The composition can also be applied to the affected area for the treatment of already inflamed skin injuries or skin diseases or for the prevention of microbial infestation of skin injuries or skin diseases. Skin injuries can be, for example, cuts or abrasions. Skin diseases can be, for example, cold sores or eczema.

The antimicrobial composition can also be used to treat hoof rot. Hoof rot is a bacterial disease of the hoof in hoofed animals, especially horses. The ray horn of the hoof is broken down by putrefactive bacteria. Treatment is usually carried out by removing the affected horn.

Treatment with the composition can reduce the bacterial infestation so that removal of the horn is not necessary.

The composition can be applied to the affected surface once or several times for treatment. For example, the composition can be applied once, twice or three times to the surface to be treated. With repeated treatment, the concentration of colloidal silver may be lower than with a single application.

In a further embodiment, the antimicrobial composition can be used in cosmetic or medical products. For example, a cream or lotion can be used as a cosmetic product. Such a cream or lotion is particularly effective against skin blemishes and pimples. For use in a cream or lotion, the concentration of colloidal silver can be reduced. For example, the cream or lotion may contain the colloidal silver in a concentration ranging from 200 ppm to 400 ppm.

In a medical product, the antimicrobial composition can be used, for example, in wound dressings. For this purpose, the wound overlay of the wound dressing can be provided with the antimicrobial composition. The composition can also be used in the form of a spray. For use in medical products, the colloidal silver may be present in a concentration in the range of 400 ppm to 1000 ppm, preferably in a concentration in the range of 500 ppm to 700 ppm.

The antimicrobial composition can also be used as a wood preservative. Wooden components, especially wooden components that are installed outside of buildings, are often affected by fungal rot due to contact with moisture. By treating with the antimicrobial composition described here, fungal infestation of the wooden component can be reduced.

It has also been shown that the use of treated irrigation water that has been mixed with the antimicrobial composition means that cut flowers stay fresh longer and wilt more slowly.

Another use of the antimicrobial composition may be the treatment of walls, ceilings and/or floors of buildings. The corresponding surfaces are pre-treated with the composition and, after drying, can be painted over with commercial paint. The pretreated surfaces have less mould growth than untreated surfaces.

In a further embodiment, the antimicrobial composition can be used to coat surfaces. Surfaces made of metal, plastic and/or glass can preferably be coated with the composition. Advantageously, objects and surfaces can also be subsequently provided with an antimicrobial layer. As only silver colloids located directly on the surface of the coating release silver ions, a continuous release of small quantities of antimicrobial silver ions can be achieved with such a coating. As a result, the coated objects and surfaces clean themselves.

The coating can be applied permanently or temporarily to the objects or surfaces. Depending on the mechanical stress on the coated surface, the coating can be repeated to ensure sufficient and consistent coating of the object or surface.

Depending on the use, additional ingredients can be added to the antimicrobial composition. The components are known from the respective speciality and are usually used for the corresponding application.

Furthermore, the invention provides a method for producing an antimicrobial composition. The method comprises the following steps:
Mixing a stabiliser capable of stabilising colloidal silver with deionised water;
optional addition of a reaction accelerator;
adding at least one water-soluble silver salt to the mixture;
neutralising the pH by adding an alkaline composition, preferably a dispersion of anionic silicon dioxide particles; and
stirring the composition to build up colloidal silver.

The use of demineralised water reduces the risk of silver compounds forming instead of colloidal silver, which precipitate in the water and sink to the bottom.

In order to accelerate the build-up of the colloidal silver particles, a reaction accelerator can be added to the process. A suitable reaction accelerator is, for example, paraformaldehyde.

Ascorbic acid can also be used as a reaction accelerator. Ascorbic acid can be used undiluted or as a diluted aqueous solution in the process. Small amounts of reaction accelerators are necessary to reduce reaction times. The reaction accelerator can be used in a concentration in the range of 0.05 g/l to 0.3 g/l, preferably in a concentration of 0.1 g/l to 0.2 g/l.

In a further embodiment, silver nitrate or silver citrate is used as the silver salt. Silver nitrate has the advantage that it is very soluble in water and therefore all the silver used is available for the production of colloidal silver. Silver citrate has the advantage that the citrate has a pH-buffering and stabilising effect on the composition.

To build up the colloidal silver particles, the silver ions of the silver salts present in the composition are reduced to elemental silver. The size and concentration of the silver particles in the composition therefore depend, among other things, on the concentration of silver ions or silver salt used in the method.

The silver salt can be used in the method in a concentration ranging from 1 g/l to 5 g/l. Preferably, the silver salt can be used in a concentration in the range of 2 g/l to 4 g/l. Preferably, the silver salt is used in a concentration of 1 g/l, 1.5 g/l, 2 g/l, 2.5 g/l, 3 g/l, 3.5 g/l, 4 g/l, 4.5 g/l or 5 g/l.

In a further embodiment, the composition has a pH in the range of 4 to 5 prior to the addition of the silver nitrate. At a pH greater than 5, the silver could precipitate as silver oxide or silver hydroxide and no colloidal silver is formed.

In a further embodiment, the pH is neutralised by the addition of a suitable alkaline composition. Suitable alkaline compositions allow a controlled increase of the pH in the composition and do not lead to the precipitation of poorly soluble silver compounds.

In a further embodiment, the composition is neutralised by the addition of a dispersion of anionic silicon dioxide particles. The use of a dispersion of anionic silicon dioxide particles is advantageous because it does not contain any interfering components that could cause the silver to precipitate as poorly soluble silver compounds. In particular, sodium hydroxide or sodium carbonate can be dispensed with to neutralise the pH of the composition, which could lead to the formation of poorly soluble silver oxide or silver carbonate. Silicon dioxide also has a stabilising effect on the colloidal silver that forms.

Dispersions with a particle diameter in the range of 5 nm-15 nm are suitable as dispersions of anionic silicon dioxide particles. Preferably, the silicon dioxide particles have an average diameter of 5 nm, 7 nm, 10 nm, 12 nm or 15 nm. In a preferred embodiment, a dispersion of anionic silicon dioxide particles is used which has an average particle diameter of 15 nm. For example, a dispersion marketed under the name Köstrosol® 1530 can be used.

The solids content of the dispersion of anionic silicon dioxide particles used can be in the range of 20 wt. %-40 wt. %. Preferably, the solids content is in the range of 20 wt. %-30 wt. %. In particular, the solids content of the dispersions is 20 wt. %, 25 wt. %, 30 wt. %, 35 wt. % or 40 wt. %.

In a further process step, the composition can be adjusted to the above-mentioned concentration of colloidal silver using demineralised water. In a further process step, a polymer dispersion and/or a surfactant can be added to the composition in the above-mentioned concentration.

To increase the reaction speed further, the process steps can be carried out at high temperatures when using agar as a stabiliser. The mixing of the stabiliser with the demineralised water can take place at a temperature in the range from 90° C. to 100° C., preferably at a temperature of 90° C. After adding the water-soluble silver salt, the composition can be stirred at a temperature in the range of 70° C. to 90° C. Preferably the composition will be stirred at a temperature in the range of 75° C. to 85° C. Preferably the temperature can be 80° C.

In a preferred embodiment, the stabiliser is agar and
the stabiliser is mixed with deionised water at a temperature in the range of 90° C. to 100° C.;
after adding the water-soluble silver salt, the composition is stirred at a temperature in the range of 70° C. to 90° C.;
the dispersion of anionic silicon dioxide particles is added until the composition has a pH in the range of 6.5 to 7; and
the composition is cooled to 40° C. with stirring to form colloidal silver.

In a further embodiment, the agar is mixed with demineralised water at a temperature in the range of 90° C. to 100° C. for a period of 1 hour to 2 hours. The time period depends on the time until the agar is completely dissolved in the water.

In a further embodiment, the composition is stirred for about 5 to 10 minutes after the addition of the water-soluble silver salt. This allows the silver salt to dissolve completely.

In a further embodiment, the dispersion of anionic silicon dioxide particles is added to the composition over a period of 3 to 5 hours. This period is necessary because the pH adjustment must be precisely controlled and therefore only small amounts of the dispersion of anionic silicon dioxide particles are added gradually. Adding the dispersion of anionic silicon dioxide particles in large volumes would cause the local pH to rise too much and poorly soluble silver compounds would form.

In a further embodiment, the composition is cooled to room temperature. The agar solidifies in the process. The composition can now be treated in such a way that the composition is pourable and has uniformly sized particles. This can be achieved, for example, by using a mixer or a disperser, which crushes the solid composition into a uniform pourable mass.

In a further embodiment, the stabiliser may be a dispersion of cationic silicon dioxide particles. A dispersion of cationic silicon dioxide particles as a stabiliser has the advantage that, when used as a coating, the transparency of the coating film is improved compared to agar. In addition, such a composition can be sprayed more easily.

Dispersions with a particle diameter in the range of 5 nm-15 nm are suitable as dispersions of cationic silicon dioxide particles. Preferably, the silicon dioxide particles have an average diameter of 5 nm, 7 nm, 10 nm, 12 nm or 15 nm. In a preferred embodiment, a dispersion of cationic silicon dioxide particles is used which has an average particle diameter of 15 nm. For example, a dispersion that is sold under the name Köstrosol® K 1530 can be used.

The solids content of the dispersion of cationic silicon dioxide particles used can be in a range of 20% by weight-40% by weight. Preferably, the solids content is in the range of 20 wt. %-30 wt. %. In particular, the solids content of the dispersions is 20 wt. %, 25 wt. %, 30 wt. %, 35 wt. % or 40 wt. %.

When using a dispersion of cationic silicon dioxide particles as a stabiliser, the process steps are carried out at a temperature in the range of 25° C. to 40° C. Temperatures above 40° C. have a destabilising effect on the dispersion of silicon dioxide particles. Due to the low temperatures, a reaction accelerator is not used.

In a further preferred embodiment, the stabiliser is a dispersion of cationic silicon dioxide particles and
the stabiliser is mixed with deionised water at a temperature in the range of 25° C. to 40° C.;
after adding the water-soluble silver salt, the composition is stirred at a temperature in the range of 25° C. to 40° C.;
the dispersion of anionic silicon dioxide particles is added until the composition has a pH in the range of 6.5 to 7; and
the composition is stirred at a temperature in the range of 25° C. to 40° C. to form colloidal silver.

In a further embodiment, the dispersion of cationic silicon dioxide particles is mixed with demineralised water at a temperature in the range of 25° C. to 40° ° C. for a period of 5 to 10 minutes.

In a further embodiment, the composition is stirred for about 5 to 10 minutes after the addition of the water-soluble silver salt. This allows the silver salt to dissolve completely.

In a further embodiment, the dispersion of anionic silicon dioxide particles is added to the composition over a period of 3 to 5 hours. This period is necessary because the pH adjustment must be precisely controlled and therefore only small amounts of the dispersion of anionic silicon dioxide particles are added gradually. Adding the dispersion of anionic silicon dioxide particles in large volumes would cause the local pH to rise too much and poorly soluble silver compounds to form.

The method described here makes it possible to produce antimicrobial compositions in which colloidal silver with particle diameters of up to 9000 nm and high concentrations can be built up and stabilised. The construction and stabilisation of colloidal silver particles with the particle sizes mentioned opens up a variety of possible uses, some of which are described here.

In one embodiment, the colloidal silver can be constructed with a particle diameter of 700 nm to 9000 nm. Preferably the particle diameter is in the range from 800 nm to 5000 nm. The particle diameter is preferably in the range from 850 nm to 3000 nm, preferably in the range from 900 nm to 2000 nm, preferably in the range from 950 nm to 1500 nm. The particle diameter of colloidal silver can be 700 nm, 800 nm, 900 nm, 1000 nm, 1100 nm, 1200 nm, 1300 nm, 1400 nm, 1500 nm, 2000 nm, 2500 nm, 3000 nm, 3500 nm, 4000 nm, 4500 nm, 5000 nm, 5500 nm, 6000 nm, 6500 nm, 7000 nm, 7500 nm, 8000 nm, 8500 nm or 9000 nm.

In a further embodiment, the colloidal silver can be built up in a concentration of over 100 ppm. Preferably in a concentration of over 200 ppm, more preferably in a concentration of over 300 ppm, preferably over 500 ppm. In one embodiment, the colloidal silver can be built up in a concentration of over 800 ppm, preferably over 1000 ppm.

In one embodiment, the colloidal silver can be built up in a concentration in the range of 100 ppm to 1500 ppm. Preferably in a concentration in the range from 200 ppm to 1000 ppm, more preferably in the range from 300 ppm to 800 ppm, preferably in the range from 400 ppm to 600 ppm. Concentrations in the range of 1000 ppm to 1500 ppm can also be built up, preferably in the range of 1200 ppm to 1300 ppm.

There are now various ways to design and further develop the framework around the current invention in an advantageous manner. Reference should be made, on the one hand, to the claims following claim 1 and, on the other hand, to the following explanation of preferred embodiments of the invention.

### EXAMPLES

To demonstrate the antimicrobial effectiveness of the composition described here, antimicrobial activity measurements were carried out in accordance with ISO 22196. The antimicrobial activity of the composition was measured against the example bacteria Escherichia coli (E. coli) and Staphylococcus aureus (S. aureus), as well as against the example fungi Candida albicans (C. albicans) and Aspergillus niger (A. niger). The reduction refers to the germ sample in relation to the corresponding reference sample without the composition. A composition containing colloidal silver at a concentration of 300 ppm was used. The results of the measurement are summarised in the following table:

| Test microbe | Reduction [%] | Log Reduction |
|---|---|---|
| E. coli | >99.99 | >4 |
| S. aureus | >99.99 | >4 |
| C. albicans | >99.99 | >4 |
| A. niger | >99.99 | >4 |

The measurements confirm the antimicrobial effectiveness of the composition. In particular, the measurements show that the composition is highly effective against different bacteria and fungi. The composition has been shown to be effective against both gram-positive and gram-negative bacteria. The composition also has antifungal activity against yeasts and moulds. All tested germs were reduced by over 99.99% compared to the reference sample.

Furthermore, the antimicrobial effectiveness of artificially aged compositions was tested after simulated 6 months and 12 months. Artificial ageing was carried out in accordance with the ASTM S 1980 standard. The same bacterial count reductions were measured for the aged compositions as above, i.e. a bacterial count reduction of >99.99% (>log 4), compared to the reference sample.

No silver ions could be detected in the composition produced using the method described here. Sodium chloride or hydrochloric acid was added to the composition for this purpose. No precipitation of sparingly soluble silver chloride was observed. Consequently, the silver in the composition is not present as silver ions and all of the silver salt has been converted.

With regard to further advantageous embodiments of the composition according to the invention, in order to avoid repetition, reference is made to the general part of the description and to the attached claims.

Finally, it should be expressly noted that the above-described embodiments of the composition according to the invention serve only to discuss the claimed teaching, but do not limit it to the embodiments.

1. An antimicrobial composition comprising colloidal silver and at least one stabiliser which stabilises the colloidal silver, wherein the colloidal silver has a particle diameter in the range 700 nm to 9000 nm.

2. The antimicrobial composition according to claim 1, wherein the stabiliser is agar and/or a dispersion of silicon dioxide particles.

3. The antimicrobial composition according to claim 1, wherein the composition further comprises at least one film-forming polymer dispersion.

4. The antimicrobial composition according to claim 1, wherein the composition further comprises at least one surfactant, preferably the surfactant is an ethoxylated fatty alcohol.

5. The antimicrobial composition according to claim 1, wherein the composition further comprises titanium dioxide.

6. The antimicrobial composition according to claim 1, wherein the colloidal silver is present in the composition in a concentration greater than 100 ppm.

7. The antimicrobial composition according to claim 1 for use in human and/or veterinary medicine, preferably for the treatment or prevention of superficial bacterial, mycotic and/or viral diseases.

8. Use of the antimicrobial composition according to claim 1 in cosmetic or medical products; as a wood preservative; for treating irrigation water for plants; for treating walls, ceilings and/or floors of buildings; or for coating surfaces, in particular for surfaces made of metal, plastic and/or glass.

9. A method for producing an antimicrobial composition comprising the steps:
mixing a stabiliser capable of stabilising colloidal silver with deionised water;
adding optionally a reaction accelerator;
adding at least one water-soluble silver salt to the mixture;
neutralising pH by adding an alkaline composition, preferably a dispersion of anionic silicon dioxide particles; and
stirring the composition to build up colloidal silver.

10. The method according to claim 9,
wherein the stabiliser is agar;
wherein the stabiliser is mixed with demineralised water at a temperature in the range of 90° ° C. to 100° C.;
wherein, after addition of the water-soluble silver salt, the composition is stirred at a temperature in the range of 70° ° C. to 90° C.;
wherein the dispersion of anionic silicon dioxide particles is added until the composition has a pH in the range of 6.5 to 7; and
wherein the composition is cooled to 40° C. with stirring to produce colloidal silver.

11. The method according to claim 9,
wherein the stabiliser is a dispersion of cationic silicon dioxide particles;
wherein the stabiliser is mixed with demineralised water at a temperature in the range of 25° ° C. to 40° C.;
wherein after addition of the water-soluble silver salt, the composition is stirred at a temperature in the range of 25° C. to 40° C.;
wherein the dispersion of anionic silicon dioxide particles is added until the composition has a pH in the range of 6.5 to 7; and
wherein the composition is stirred at a temperature in the range of 25° C. to 40° ° C. to form colloidal silver.
